(19) United States
(12) Patent Application Publication
(10) Pub. No.: US 2009/0062782 A1
(43) Pub. Date: Mar. 5, 2009

(54) Laser Delivery Apparatus With Safety Feedback System

(76) Inventor: Joe Denton Brown, Panama City, FL (US)

Correspondence Address: BACON & THOMAS, PLLC, 625 SLATERS LANE, FOURTH FLOOR, ALEXANDRIA, VA, 22314-1176, US

(21) Appl. No.: 12/047,819

(22) Filed: Mar. 13, 2008

Related U.S. Application Data
(60) Provisional application No. 60/906,513, filed on Mar. 13, 2007.

(51) Int. Cl. A61B 18/20

(52) U.S. Cl. 606/15

(57) ABSTRACT

An apparatus and method for delivery of energy to a tissue within a patient includes optical or electrical feedback to detect effects of overheating and/or burning of tissues, bodily fluids, or the apparatus itself, circuitry for controlling the delivery apparatus, and/or indicators to facilitate operator control. Provision is also made for fiber position detection to facilitate manual or automatic control of fiber positioning or withdrawal relative to or through an introducer.

This application claims the benefit of U.S. Provisional Patent Application Ser. No. 60/906,513, filed Mar. 13, 2007.

### BACKGROUND OF THE INVENTION

1. Field of the Invention

This invention relates to apparatus for delivering energy to a tissue. The apparatus includes a safety feedback control unit, and optionally, a manual or automatic fiber position, laser activation, and rate of pullback controller.

The apparatus of the invention is applicable, by way of example, to treatment of blood vessels using endovascular techniques for delivering laser energy.

2. Description of Related Art

A. Safety Feedback Control System and Method

U.S. Pat. No. 5,098,427 discloses a surgical instrument for delivering fiber-optically guided radiation to biological tissue that prevents overheating by detecting visible light (light in the visible spectral region between 0.3 and 0.9 μm) resulting from pyrolytic glowing of partially carbonized tissue, and controlling the laser accordingly.

Copending U.S. patent application Ser. No. 11/510,691, filed Aug. 28, 2006 and incorporated by reference herein, discloses a surgical instrument for delivering fiber-optically guided radiation to biological tissue, in which (I) an introducer sheath and or fluids in the sheath serve as a waveguide for radiation, which does not need to be in the visible range, generated by burning tissues, (ii) or the clarity of fluid in the waveguide is analyzed to check for proper flushing or to indirectly detect effects of overheating.

Copending U.S. patent application Ser. No. 11/714,785, filed on Mar. 7, 2007 by the present inventor discloses a variation of the arrangement disclosed in U.S. patent application Ser. No. 11/510,691, in which the feedback originates from a thermocouple or other heat sensing device that outputs an electrical signal indicative of overheating or burn back. In addition, copending U.S. patent application Ser. No. 12/______, filed Mar. 12, 2008 in the name of Joe D. Brown, discloses further variations and improvements to the feedback arrangement disclosed in U.S. patent application Ser. No. 11/510,691, control of the laser based on the relative position of the fiber and an introducer or catheter.

The present invention can use any or all of the above feedback arrangements to control the laser or fiber position. It can be used in connection with a method for treating varicose veins, such as the one disclosed in U.S. Pat. No. 6,398,777 or in other surgical procedures.

B. Fiber Position and Rate of PullBack Control

U.S. Pat. No. 6,981,971 discloses fiber position control based on markings at a distal end of an introducer, as well as markings on the fiber. The markings enable the introducer and fiber to be withdrawn at a controlled rate. The present invention also uses fiber markings, but instead of pulling back both the introducer and fiber, the present invention enables the position of the fiber to be controlled relative to the introducer based on markings on the fiber that can be read through the introducer. Conveniently, the markings may be at the entrance side of the introducer rather than at the distal end, enabling use of a fiber position and rate of pullback controller to enable convenient positioning and withdrawal of the fiber. The fiber position and rate of pullback controller can also control laser activation through a single handheld device.

C. Examples of Problems Solved by Invention

Among the problems solved by the invention is the problem that contact between the fiber tip 4 and blood 5 in the vein can cause overheating and burn back of cladding and other buffer materials on the fiber tip, which can damage the fiber cladding, as well as continued lasing, charring or carbonization, which can weaken fiber integrity and have negative consequences for both the efficacy of the surgical procedure and patent recovery. For example, exposing the silica core of a fiber can allow carbonization to the sides of the fiber tip making it weak with the possibility of falling off into the vein. Furthermore, carbonization forming on the distal tip can locally heat the distal fiber tip surface to extreme temperatures sufficient to enough to cause the fiber to start absorbing infrared radiation, thereby causing a thermal runaway that could perforate the vein wall. Still further, charring and/or other effects of overheating or thermal runaway can directly cause negative effects on the patient, such as operative or post-operative pain and/or toxic reactions to compounds resulting from burning or vaporization of materials such as Teflon™. Finally, if the burn back exposes the surfaces on the side of the fiber, then energy is stolen from the core, making the power density lower and affecting the treatment.

In addition to preventing negative effects resulting from burn back, the invention provides improved control of fiber position. This is useful not only during varicose vein treatment, but also in numerous other applications, such as urological applications. For example, in applications involving stone management, the doctor will often pull the fiber accidentally into the scope while lasing, thereby destroying the scope's working channel. By providing detection on the fiber and viewing the markings at the entrance of the scope rather than in the introducer hub, as disclosed in U.S. Pat. No. 6,981,971, the position of the fiber can easily be determined and controlled by a handheld position controller, reducing the potential for errors.

Furthermore, a display of fiber distal position can be superimposed on images used in the stone surgery. In that case, a secondary feature would be that the fiber could now be used as a measuring “stick” to determine the size of the stone. Often a surgeon picks too large a stone to pull out with a basket and ends up getting stuck while trying to remove the stone from the body (typically the ureter). This is a major problem for the urologists, which is eliminated by using an image of the fiber to judge the size of a stone.

### SUMMARY OF THE INVENTION

The invention provides an apparatus for delivery of energy to a tissue within a patient, in which damage to the energy delivery device and/or harm to the patient is minimized by using optical or electrical feedback to detect effects of overheating and/or burning of tissues, bodily fluids, or the apparatus itself, and for controlling the delivery apparatus accordingly.

The invention may utilize either an external or internal control unit and a catheter/introducer with a hub for a feedback signal carrier. The feedback signal carrier may be an optical fiber, in the case of optical feedback, or an electrical connection.

In one preferred embodiment, the control unit includes an input from an optical feedback fiber optic cable which transmits optical feedback signals corresponding to the ones utilized in patent application Ser. No. 11/510,691. The optical feedback signal is separated into a first wavelength spectrum corresponding to the aiming beam, the attenuation of which indicates build-up of contaminants such as charring on the fiber tip and/or inadequate flushing, and a second wavelength spectrum including infrared wavelengths that directly detect overheating or burning.

If either detector detects a dangerous condition, then a signal is output to the laser control. If the control unit is external to the fiber control unit, then the warning signal may be transmitted to the door interlock conventionally provided in the laser and the control unit may include an audible or visual warning indicator. Alternatively, the necessary detection circuitry and components may be internal to the control unit.

The apparatus of the invention may also include a manual or automatic fiber position, laser activation, and rate of pullback indicator/control unit that utilizes fiber markings at the entrance side of the introducer to determine fiber position. The indicator/control unit may include a laser trigger, a display of the laser power setting, for example in watts, a display showing the results of a calculation of the laser energy used, and a display of the rate at which the fiber is manually fed into the or withdrawn from the catheter/introducer. Feeding of the fiber may be carried out by hand or by a motorized or automated feed unit.

### BRIEF DESCRIPTION OF THE DRAWINGS

FIG. 1 is a schematic drawing of a feedback control unit according to the present invention.

FIG. 2 is a diagram showing components of an external feedback system according a preferred embodiment of the present invention.

FIG. 3 is a diagram showing an internal feedback system according to another preferred embodiment of the present invention.

FIG. 4 is a diagram showing the addition of a fiber position, laser activation and manual rate of pullback indicator/control unit to the internal feedback system of FIG. 3.

FIG. 5 is a top view showing a manual version of the indicator/control unit of FIG. 4.

FIG. 6 is a top view showing an automatic version of the indicator/control unit of FIG. 4.

FIGS. 7 and 8 show two different set-ups for the control unit of FIG. 4.

FIG. 9 illustrates operation of the indicator/control unit of FIG. 5, including use of fiber markings to indicate the end of the fiber.

FIGS. 10 and 11 respective show fiber tip arrangements for fibers with an all-silica core/cladding and fibers with a glass core, including a thermocouple measuring tip and a protective sheath.

### DETAILED DESCRIPTION OF THE PREFERRED EMBODIMENTS

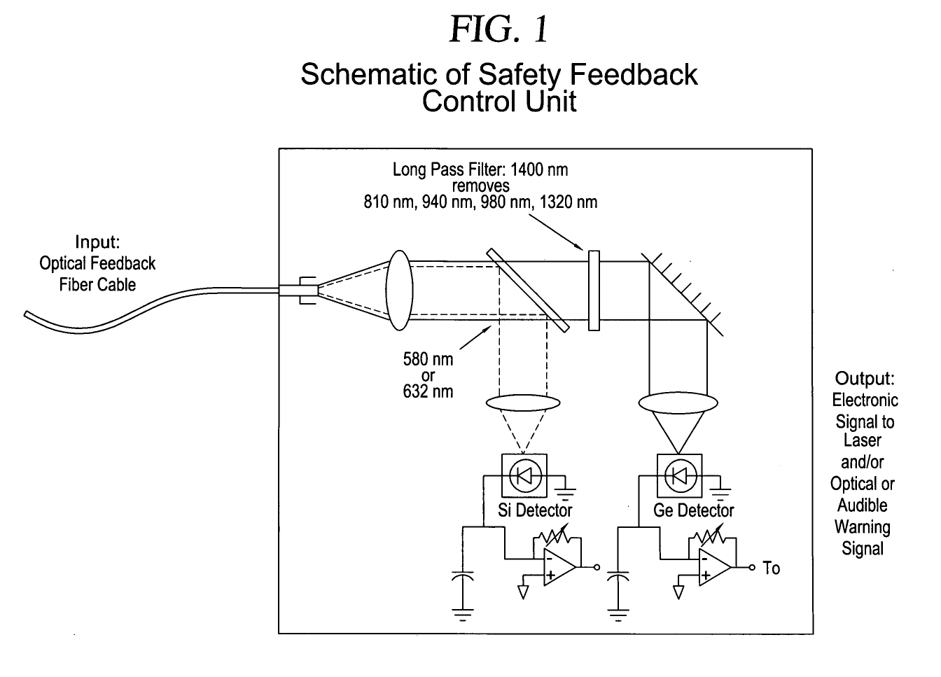

The feedback control shown in FIG. 1 is arranged to be used in an external feedback control unit. It includes an optical fiber cable input, through which are transmitted optical feedback signals indicative of charring or overheating. A lens collimates the signal and feeds it to a low pass filter that eliminates wavelengths above visible. A half-silvered mirror or functionally equivalent optical element separates the remaining wavelengths into visible and infrared wavelengths for respectively detecting attenuation of the aiming beam and infrared light emitted during burn up. The outputs of the detectors are supplied to threshold detectors in the form of comparators that output an electronic signal when the optical detectors output a signal that exceeds a threshold. The optical detectors are illustrated as an Si detector and a Ge detector, though the exact wavelengths shown in FIG. 1 may be varied depending on the nature of the feedback signal, as may the optical elements, detectors, and associated circuitry.

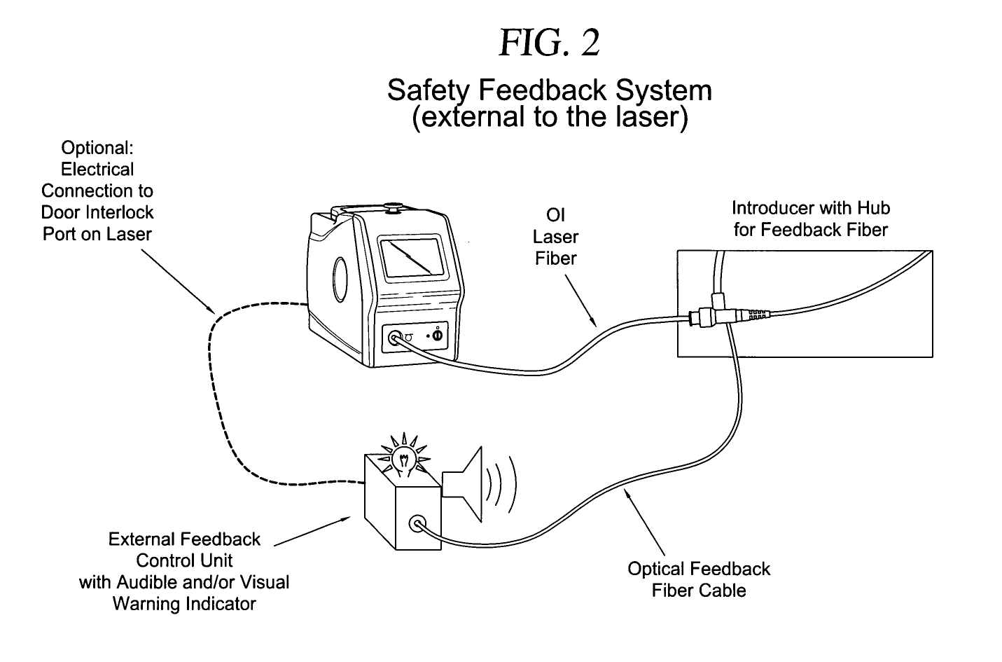

The feedback control unit circuitry illustrated in FIG. 1 may be used to supply a warning signal to the laser controller, for example, via the door interlock included on many controllers. FIG. 2 shows the external feedback control unit with an audible and/or visual warning indicator responsive to the outputs of the comparators, a hub through which the laser output is transmitted to a delivery fiber, and through which optical feedback signals are fed to the optical feedback fiber cable. The optical feedback may be through the illustrated feedback fiber, the catheter/introducer may act as a waveguide, the optical feedback may propagate through fluid in the catheter/introducer, or the feedback may be through the cladding of the delivery fiber. Alternatively, though not illustrated, the feedback may be in the form of electrical signals from an optical detector in the catheter/introducer, a thermocouple, thermistor, or other heat detecting device in the catheter/introducer, or a photodetector in the catheter/introducer.

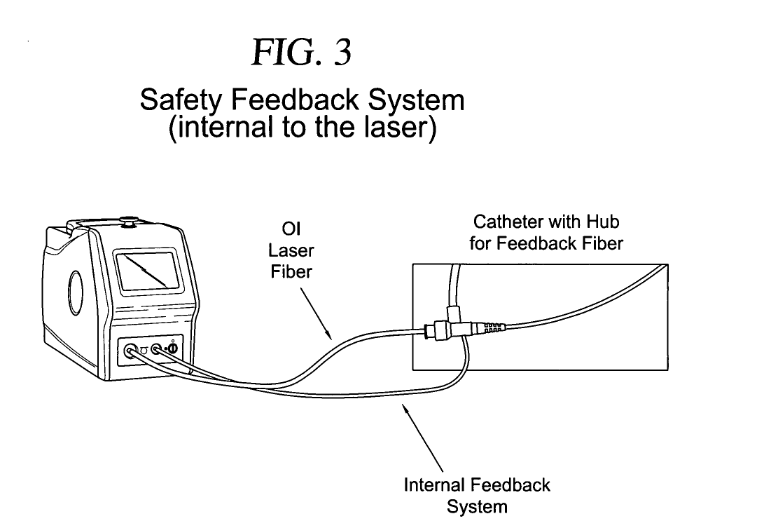

As illustrated in FIG. 3, the feedback may be supplied directly to internal circuitry in the controller.

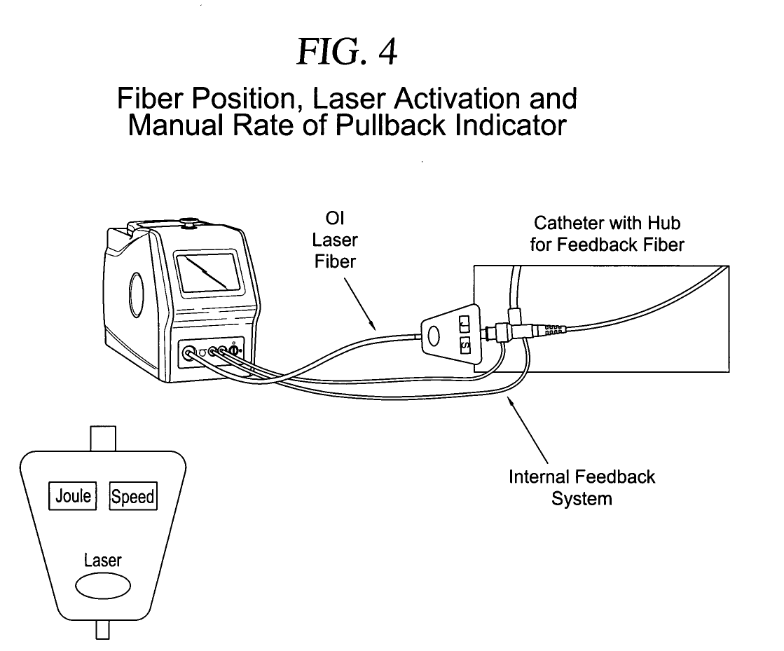

As illustrated in FIG. 4, the feedback controller may be used with a fiber position, laser activation, and manual rate of pullback indicator/controller attached to the hub of the catheter/introducer that utilizes markings on the fiber to determine and display fiber position. The pullback indicator/controller includes a button or other control for manually triggering the laser, a display of laser power setting of the laser (Watts), a display of calculated laser delivery energy (Joules), and a display of the feed rate at which the fiber is manually fed through the indicator. Connection port C1 connects to the laser trigger port, usually a foot switch port, to enable the button to trigger the laser.

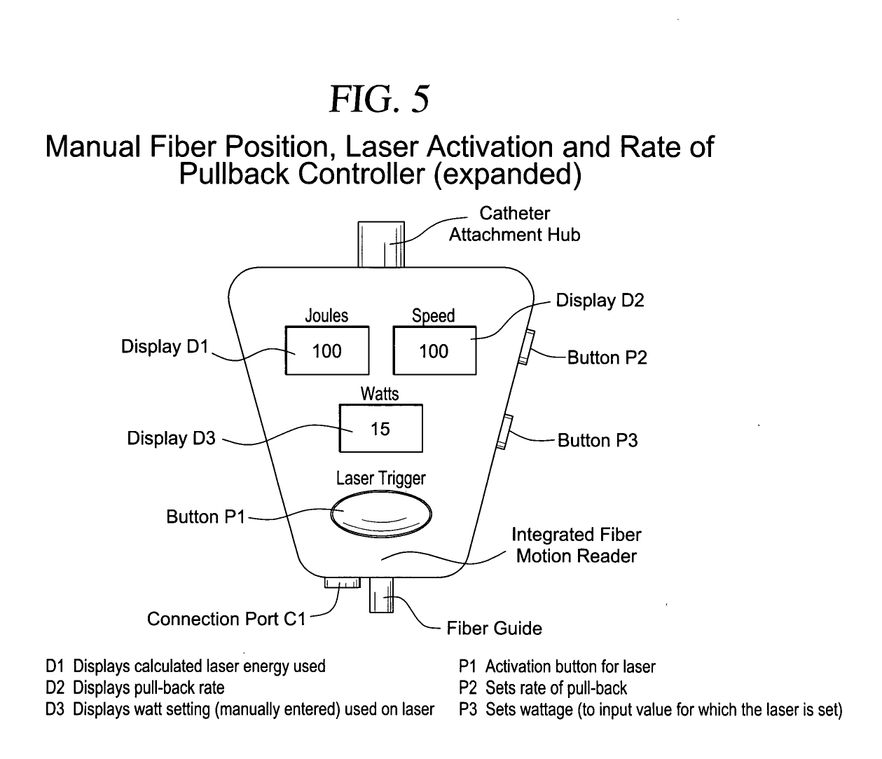

FIG. 5 shows the same arrangement as FIG. 4, but with a motorized or automated fiber feed, in which a second connection port C2 is included for receiving feedback signals for transfer, through a cable connected to port C1, to the laser.

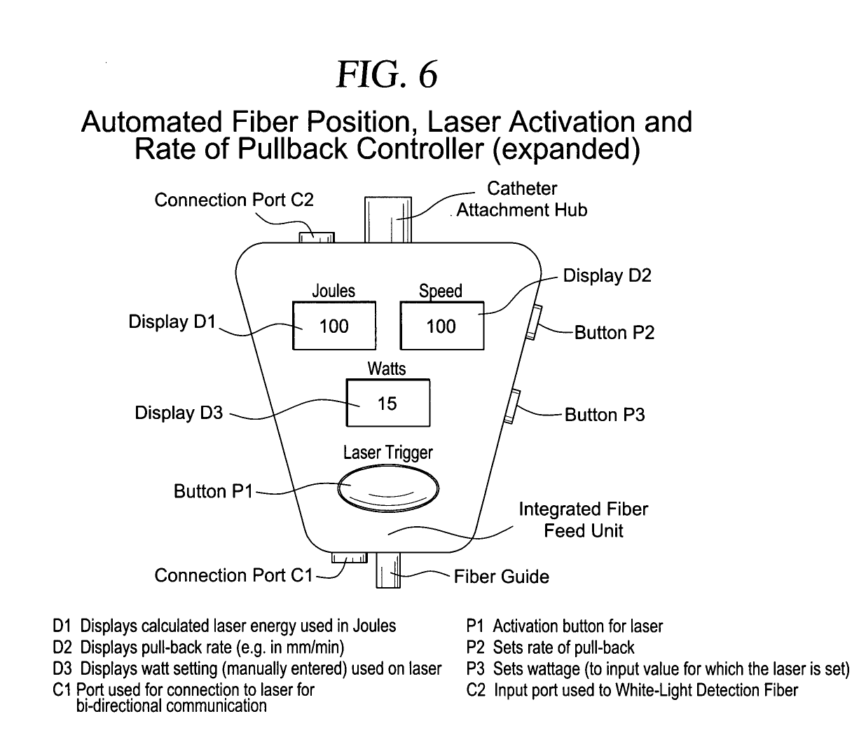

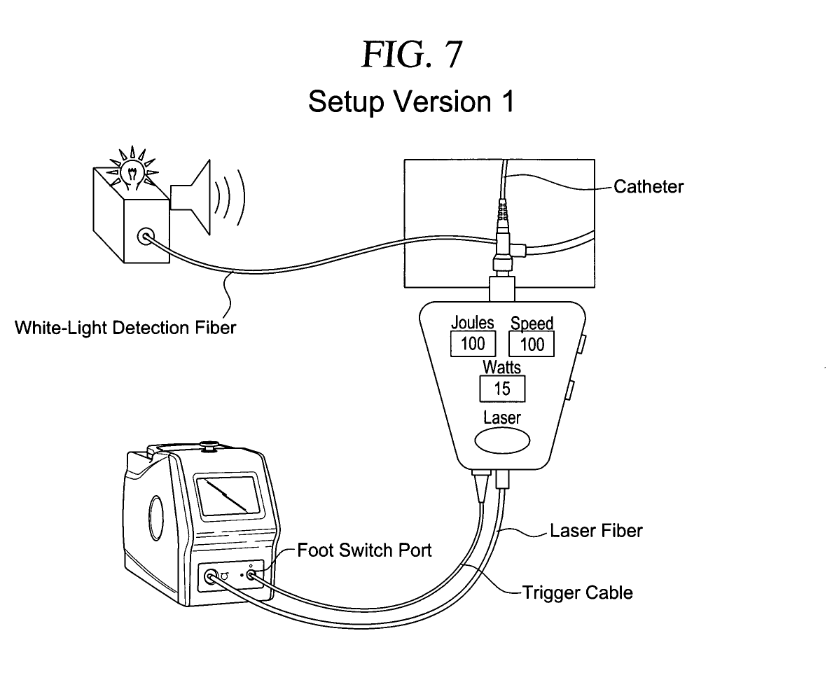

FIG. 6 shows a first setup in which the hub is connected to an external feedback control and the pullback indicator/controller is connected to the foot switch port for triggering. FIG. 7 shows a second setup in which the feedback cable is connected to port C2 and communicated through port C1, along with the trigger control signal, to the laser for automated control.

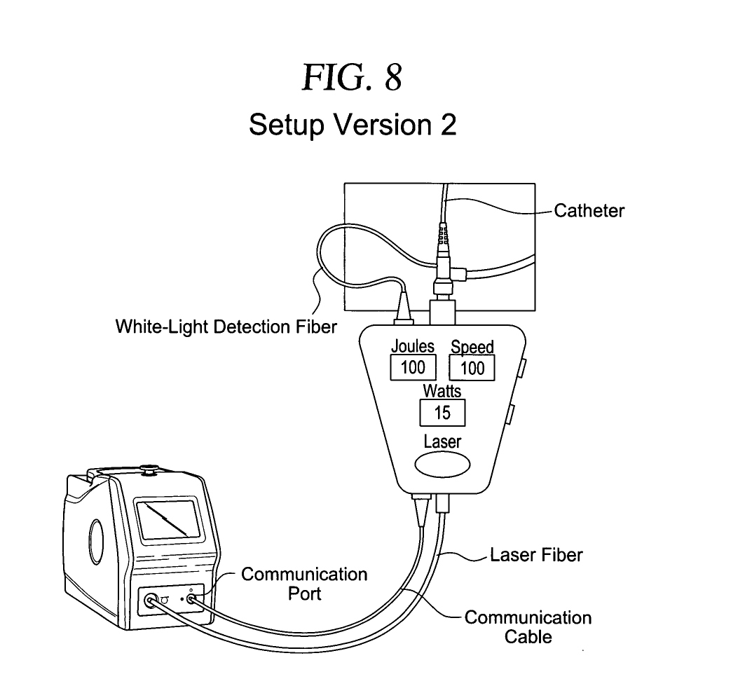

FIG. 8 shows a pullback indicator/controller of the type illustrated in FIG. 5, in which the fiber is pulled back through the stationary catheter/introducer until a red fiber section indicates reaching the end of the fiber, at which time pull back is stopped. A yellow fiber section may be used to indicate the approaching fiber end before the end is actually reached.

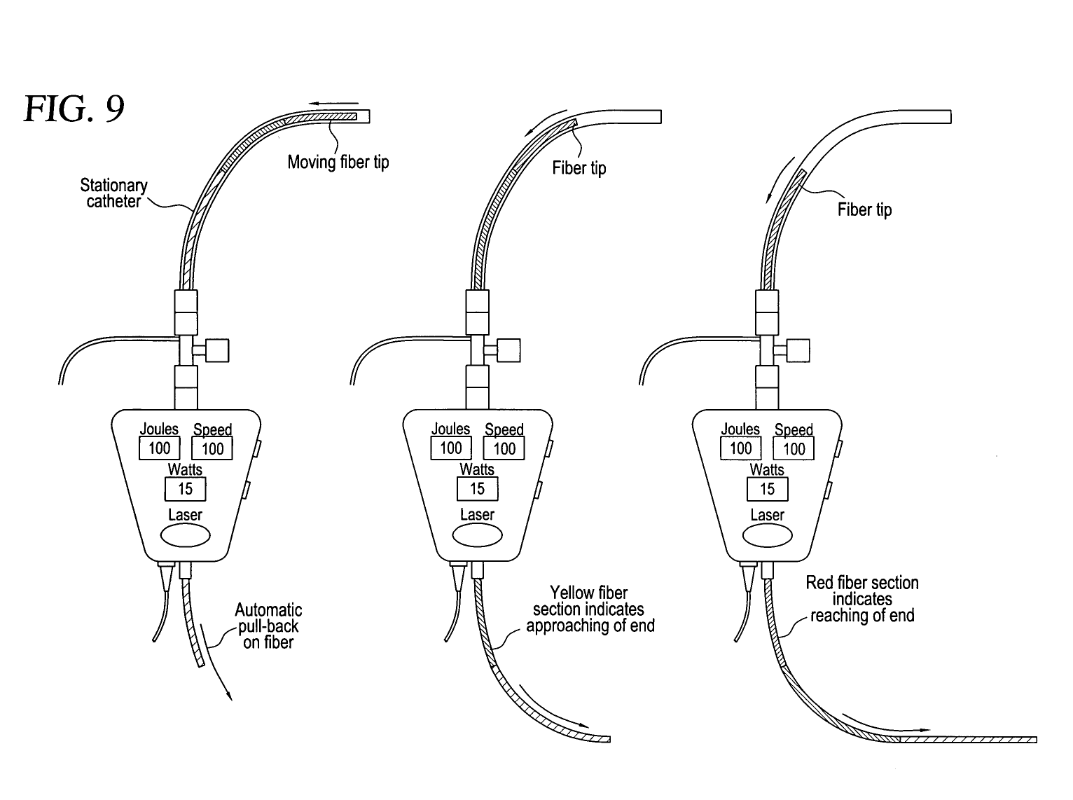

FIG. 9 illustrates use of the pullback indicator controller to pull back the fiber through a stationary introducer or catheter, including passage of marked fiber sections through the controller to provide information or feedback concerning the approaching fiber tip.

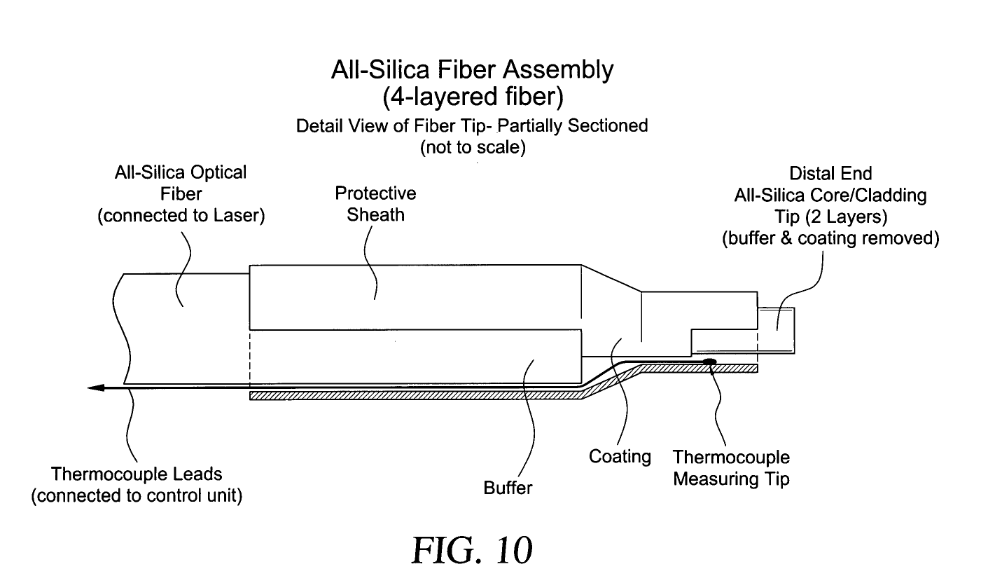

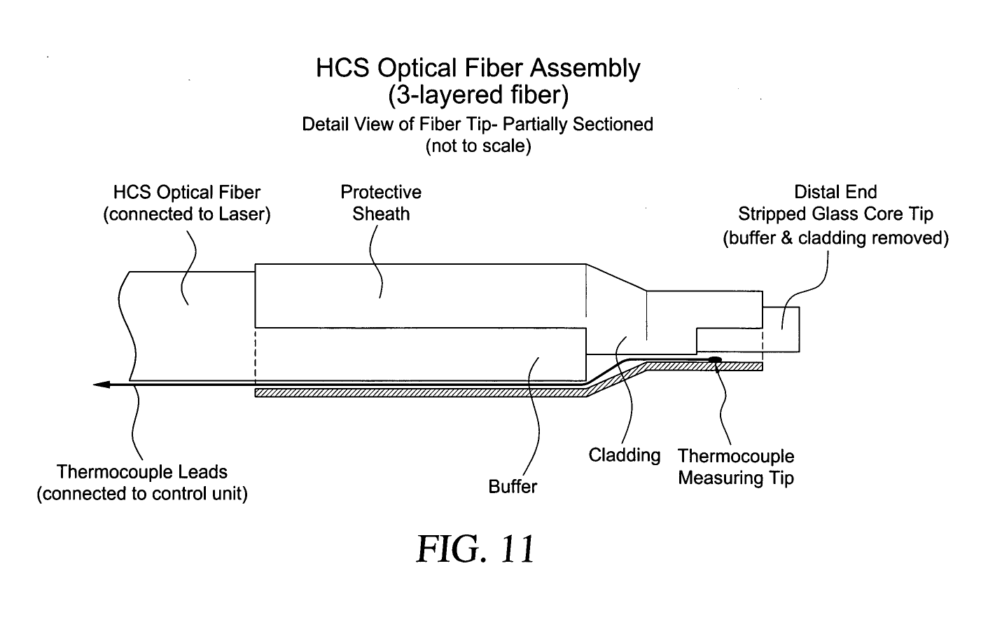

FIGS. 10 and 11 show respective fiber tip arrangements for a fiber with all silica core/cladding and a fiber with a glass core. Surrounding the fiber is a protective sheath that can be made of a reflective metal to prevent burning of the tip and to enable location of the fiber tip, and/or display of fiber tip size and position relative to, for example, a stone in the urinary tract of a patient. Also shown in FIGS. 10 and 11 is a thermocouple for temperature feedback, as described above.

Although FIGS. 10 and 11 show specific fiber and tip configurations, it will be appreciated by those skilled in the art that the invention is in general not limited to a particular fiber construction or composition, or to a particular tip configuration/shape.

Having thus described a preferred embodiment of the invention in sufficient detail to enable those skilled in the art to make and use the invention, it will nevertheless be appreciated that numerous variations and modifications of the illustrated embodiment may be made without departing from the spirit of the invention, and it is intended that the invention not be limited by the above description or accompanying drawings, but that it be defined solely in accordance with the appended claims.

What is claimed is:

1. A control unit for laser delivery apparatus including a laser, an optical fiber for delivering energy from the laser to a treatment area of a patient, and a feedback signal carrier for carrying a signal from the treatment area back to the control unit, said control unit comprising:
signal separation means for separating said feedback signal into a first signal whose attenuation indicates build-up of contaminants at a distal end of said optical fiber, and a second signal indicative of a temperature of said treatment area;
a first detector for detecting said first signal; and
a second detector for detecting said second signal.

2. A control unit as claimed in claim 1, wherein said feedback signal is an optical signal, wherein said first signal is a first wavelength spectrum corresponding to an aiming beam, the attenuation of which indicates build-and said build-up of contaminants caused by charring of a tip of said fiber, and wherein said second signal is a second wavelength spectrum including infrared wavelengths that directly detect overheating or burning in said treatment area.

3. A control unit as claimed in claim 2, wherein feedback signal is separated by a beam splitter, said first wavelength spectrum being detected by said first detector and said second wavelength spectrum being detected by said second detector.

4. A control unit as claimed in claim 3, wherein said beam splitter is a half-silvered mirror

5. A control unit as claimed in claim 1, wherein if either of said first and second detectors detects a dangerous condition, then a warning signal is output to a laser control.

6. A control unit as claimed in claim 5, wherein said warning signal is transmitted to a door interlock provided in said laser control.

7. A control unit as claimed in claim 5, wherein said control unit includes an audible or visual warning indicator responsive to said warning signal.

8. A fiber position controller for use in laser delivery apparatus including a laser, an introducer, and an optical fiber that passes through said introducer for delivering energy from the laser to a treatment area of a patient, said fiber position controller comprising:
a reader for fiber markings on a second of said fiber that extends from an entrance side of said introducer, said fiber markings being indicative of a position of said fiber relative to said introducer, and said reader outputting a fiber position signal indicative of said fiber position.

9. A fiber position controller as claimed in claim 8, further comprising a display responsive to said fiber position signal for displaying said fiber position.

10. A fiber position controller as claimed in claim 9, wherein said display further includes a display of a power setting of said laser.

11. A fiber position controller as claimed in claim 9, wherein said display further includes a display of calculated laser delivery energy.

12. A fiber position controller as claimed in claim 9, wherein said display further includes a display of a rate at which the fiber is manually fed through the fiber position controller, said rate being calculated based on the fiber position signal.

13. A fiber position controller as claimed in claim 8, wherein said fiber is fed manually through said controller.

14. A fiber position controller as claimed in claim 8, further comprising an automatic fiber feed.

15. A fiber position controller as claimed in claim 14, wherein the automatic fiber feed includes a motor.

16. A fiber position controller as claimed in claim 8, further comprising a trigger for controlling an output of said laser.

17. A fiber position controller as claimed in claim 16, wherein said motor is controlled by a foot switch connected to the fiber position controller.

18. A fiber position controller as claimed in claim 8, wherein said fiber position controller is arranged to receive a feedback signal from a feedback signal carrier, said feedback signal indicative of overheating or burning at a distal end of said optical fiber.

19. A fiber position controller as claimed in claim 18, further comprising a laser control unit, said laser control unit comprising:
signal separation means for separating said feedback signal into a first signal whose attenuation indicates build-up of contaminants at a distal end of said optical fiber, and a second signal indicative of a temperature of said treatment area;
a first detector for detecting said first signal; and
a second detector for detecting said second signal.

20. A fiber position controller as claimed in claim 19, wherein said feedback signal is an optical signal, wherein said first signal is a first wavelength spectrum corresponding to an aiming beam, the attenuation of which indicates build-and said build-up of contaminants caused by charring of a tip of said fiber, and wherein said second signal is a second wavelength spectrum including infrared wavelengths that directly detect overheating or burning in said treatment area.

21. A fiber position controller as claimed in claim 18, wherein said feedback signal controls an output of said laser.

22. A fiber position controller as claimed in claim 18, wherein said feedback signal is an electrical signal output by a thermocouple.

23. A fiber position controller as claimed in claim 8, wherein a distal end of said fiber is covered by a protective sheath made of a reflective material to enable location of the fiber tip.

24. A fiber position controller as claimed in claim 23, further comprising means for displaying, by detecting said protective sheath, a fiber tip size and position relative to a pathological obstruction in said patient.

25. A fiber tip assembly, comprising a protective sheath at a delivery end of an optical fiber, said protective sheath being reflective to enable location of the fiber tip during a therapeutic laser treatment procedure, and display of a fiber tip size and position relative to a pathological obstruction in a patient.

26. A fiber tip assembly as claimed in claim 25, wherein said fiber tip assembly further includes a thermocouple for measuring a temperature in a treatment area.
